(19) United States
(12) Patent Application Publication
(10) Pub. No.: US 2018/0243121 A1
(43) Pub. Date: Aug. 30, 2018

(54) Foot orthosis with comprehensive method for correcting deformities of the transverse arch of the foot in cases of static transverse flatfoot compounded by hallux valgus, with possible preventive and post-operative applications.

(71) Applicants: Jacek Marek Dygut, Warsaw (PL); Monika Weronika Piwowar, Cracow (PL)

(72) Inventors: Jacek Marek Dygut, Warsaw (PL); Monika Weronika Piwowar, Cracow (PL)

(21) Appl. No.: 15/444,448

(22) Filed: Feb. 28, 2017

(51) Int. Cl. A61F 5/01

(52) U.S. Cl. CPC A61F 5/0127; A61F 5/019

(57) ABSTRACT

The proposed method is an innovative approach to correcting orthopedic deformities. It involves gradual manual mobilization of contracture soft tissues and diminutive foot joints by a physiotherapist, followed by mechanical reinforcement of the resulting effect by an orthosis which depresses and push the Ist, IVth and Vth metatarsal bones while elevating or actually blocking the fall the immobile IInd and IIIrd metatarsal bones according to the “three forces” rule. Correction transverse arch foot runs simultaneously with the correction of hallux valgus (if necessary). The propose method comprises sequentially applied passive redression (manual treatment), and a follow-up with the use of a specially designed orthosis (mechanical treatment). The method is suitable for patients undergoing preparation for corrective HV surgery and for post-operative HV. Method can be used preventively e.g. in women who frequently wear high-heel shoes and in for those who need to remain standing for prolonged periods of time.

### FIELD OF INVENTION

The current invention generally relates to correcting orthopedic deformities. In particular the invention concerns the treatment for restoring the physiological shape of the transverse arch of the foot by correcting static transverse flatfoot and associated hallux valgus by applying a sequential process where both components act together in order to produce the desired therapeutic effect (manual and mechanical—with the use of a specially designed orthosis).

### BACKGROUND OF THE INVENTION

The focus of the invention is a comprehensive treatment method for restoring the physiological shape of the transverse arch of the foot by correcting two of the most commonly occurring orthopedic deformities: static transverse flatfoot and associated hallux valgus. More specifically, the invention comprises a system and a protocol for restoring the transverse arch of the foot via remediation of forefoot deformities: hallux valgus (bunion), mallet toes deformity and claw toes, through manual and/or mechanical depression of the Ist, IVth and Vth metatarsal bones along with a counterforce applied to the physiologically immobile IInd and IIIrd metatarsal bones, achieved with the use of a customized orthosis. The aforementioned procedure is carried out sequentially (by gradual stretching of contracture) until the required therapeutic effect is obtained, and can be extended with a set of physiotherapeutic exercises (electrostimulation and massage with mobilize foot muscles, tendons, joints, ligaments etc. at each stage of the process).

Static transverse flatfoot and hallux valgus are among the most frequently occurring orthopedic cases. The underlying risk factors are quite diverse (they include gender, age, genetic background, comorbidities and choice of footwear), while the associated statistical data is characterized by significant spread and an abundance of outliers.

In spite of many decades of research and therapeutic developments, the causes of orthopedic malformities such as flatfoot or transverse flatfoot and hallux valgus (HV), are not fully explained. It is generally accepted that the appearance of HV usually leads to static flatfoot and vice versa—the onset of static flatfoot leads to further deformations, which typically include HV. Most often, the so-called transversal arch collapse is a link connecting both defects constituting the intermediate stage of their overlapping. However, there are observed cases where hallux valgus deformity is a direct consequence of transversal arch collapse even with proper shape of the longitudinal arch (dancers, sprinters, etc.).

Early-stage patients are usually advised to undergo noninvasive treatment with orthoses or shoe inserts. Such treatment, while effective in alleviating pain, often produces unsatisfactory therapeutic outcomes. Medical literature is replete with evidence that orthoses and casts provide only meager benefits in treating foot deformities. Consequently, many researchers point to invasive treatment as the preferable option. Such treatment is not, however, free of disadvantages: while yielding major cosmetic benefits, it often fails to address the underlying causes of pathological changes and the resulting symptoms (including pain).

To-date methods for corrective treatment of hallux valgus typically employ orthoses with retrocapital cushions (also known as pelotes). They retract the toe medially without concern for the shape of the transverse arch (in individuals where the arch is flattened or reversed). Attempts to reconstruct the transverse arch have heretofore been limited to inserts which elevate the IInd and IIIrd metatarsal. The remaining metatarsal bones are depressed gravitationally; however this only occurs under load and is only applicable at early stages of malformation in cases of insufficient muscular or ligament strength, i.e. during activity. Such inserts offer no benefits while the patient is resting and are of little use in treating severe deformities—during contractions, where additional forces depressing the Ist, IVth and Vh metatarsal while elevating the physiologically immobile 2nd and 3rd metatarsal are required.

The presented method proposes an innovative manual/mechanical approach to correcting orthopedic deformities. The method involves gradual manual mobilization of contracture soft tissues and deminutive foot joints by a physiotherapist, followed by mechanical reinforcement of the resulting effect by an orthosis which depresses and push the Ist, IVth and Vth metatarsal bones while elevating or actually blocking the fall the immobile IInd and IIIrd metatarsal bones. Depending on the degree of deformity and the expected therapeutic outcome, the proposed method may involve manual intervention and the use of an orthosis which is meant to enhance and preserve the result of manual intervention. In some cases, the orthosis may be applied directly, skipping the manual step—this applies particularly to patients who are not suffering from any musculoskeletal pathologies and in whom the stabilizing foot muscles have been weakened e.g. by improper footwear (such as high heels), as well as patients recovering from hallux valgus (HV) surgery. In most cases, however, it is expected that manual intervention and mechanical correction (orthosis) will be applied iteratively, at regular intervals, to achieve synergy.

In the authors' experience, the proposed manual/mechanical approach can be used in the following situations:
treatment of feet affected by non joint contracture and contracture of joints and joint capsules, as well as in preparation for surgical treatment of deformities (e.g. hallux valgus)
prophylactically (preventively), in patients who do not suffer from permanent deformities (wearers of non-physiological footwear such as high heels, or persons who need to remain standing for prolonged periods of time), and in postoperative patients where such intervention might enhance the effects of surgery.

### BRIEF SUMMARY OF THE INVENTION

Existing treatment options—both noninvasive (shoe inserts and orthoses) and invasive (surgical treatment)—do not provide a complete cure, i.e. do not produce effects which would approximate physiologically normal conditions. The proposed manual/mechanical method is based on an assessment of the shortcomings of existing treatments (such as HV corrective surgery). It comprises passive redression (manual treatment), where the physiotherapist manually overcomes the soft tissue contracture, and a follow-up with the use of a specially designed orthosis (mechanical treatment). Manual/mechanical treatment should be replenishment by exercises and electrostimulation designed to strengthen foot muscle. This interplay of manual and mechanical factors leads to restoration of the approximate physiological shape of the transverse arch by depressing the first, fourth and fifth metatarsal bones while elevating rather supporting the second and third metatarsal bones. Based on a series of analyses and pilot studies the authors suggest the use of the manual/mechanical approach in patients suffering from a broad range of forefoot deformities, except those in whom irreversible degenerative changes in osseous tissues have led to muscle contractures and pathological tension in the fascia, ligaments and joint capsules. The proposed method is also suitable for patients undergoing preparation for corrective HV surgery as well as for post-operative HV patients where it may enhance the effects of said surgery. Positive results have also been reported when applying the orthosis prophylactically (preventively) (e.g. in women who frequently wear high-heel shoes and in individuals who need to remain standing for prolonged periods of time).

### BRIEF DESCRIPTION OF THE DRAWINGS

FIG. 1 Overview of the therapeutic procedure. The procedure begins with a diagnosis of forefoot deformities (1). If a deformity of the transverse arch is diagnosed (Yes), the patient undergoes manual redression (2), followed by mechanical redression (3) (administered in the supine or sitting position for as long as the physician sees fit). If no corresponding deformity is diagnosed (No), no manual/mechanical therapy can be applied (End of treatment (5)). Following redression the patient is advised to undergo a series of strengthening exercises and/or electrostimulation (4). The procedure is repeated until the correct shape of the transverse arch is restored.

FIG. 2 Overview of the prophylactic (preventively) procedure. The procedure begins with a diagnosis of foot deformities (Muscle failure or postoperative stage (6)). If weakness of the musculoskeletal system is evident or the patient has recently undergone corrective surgery, e.g. for HV (Yes), mechanical redression is recommended (7) (administered in the supine or sitting position, for as long as the physician sees fit). If the aforementioned condition is not met (No), the procedure ends (End of treatment (9)). Following redression the patient is advised to undergo a series of strengthening exercises and/or electrostimulation (8). The procedure is repeated until a satisfactory effect has been be obtained.

FIG. 3 The three-force (F₁, F₂, F₃) rule, as employed by the corrective orthosis. Depression of the first, fourth and third metatarsal (11), (12) with a counterforce applied to the second and third metatarsal bones (10).

FIG. 4 Design of the mechanical orthosis: top-down projection (top) and cross section (down). 13—foot; 14—adjustable flanking clamps; 15—semi-annular elastic clamp; 16—bottom fastening screw handle; 17, 18—bottom fastening screw housing; 19—bottom fastening screw; 20—bottom fastening screw tension spring; 21—top fastening screw handle; 22, 23—top fastening screw housing; 24—top fastening screw; 25—top fastening screw tension spring; 26—foot elevator; 27—adjustable foot elevator support; 28—hallux abductor pelote (straightening function); 29—semi-annular pelote mount; 30—adjustable pelote mounting arm; 31—pelote mount with arm length adjustment capability; 32—pelote tension control screw; 33—pelote screw head handle.

### DETAILED DESCRIPTION

The manual/mechanical method for correcting static transverse flatfoot (often compounded by hallux valgus) is a sequential process where both components act together in order to produce the desired therapeutic effect, i.e. restoration of the approximate physiological transverse arch of the foot. By applying the proposed manual/mechanical method the symptoms of hallux valgus can be mitigated and the associated pain alleviated.

The proposed approach acknowledges certain aspects of existing therapeutic solutions for treatment of foot deformities; however these aspects are repurposed in a new context. First of all, the therapeutic procedure employs a specially-designed corrective orthosis, which bases on the so-called “three forces” principle—or, in other words, provides three points of application of antagonistic forces F₁ vs F₂ and F₃ (10,11,12 respectively).

The manual/mechanical system is targeted at patients suffering from a broad range of forefoot deformities, except those in whom irreversible degenerative changes in osseous tissues have led to muscle contractures and pathological tension (contractures) in the fascia, ligaments and joint capsules. It can also be applied prophylactically (preventively) in patients whose footwear does not promote physiologically sound action of tarsal muscles and bones (e.g. high heels). Finally, it may constitute a preparatory step in surgical treatment of trensverse flatfoot or HV, as well as a means of preserving the resulting therapeutic effects.

Prior to administration of the proposed manual/mechanical protocol, patients—in whom the type and degree of orthopedic deformities have previously been assessed—can be advised to undergo “tissue contracture mitigation” procedures, i.e. massage, heating (balneological treatment), potassium iodide iontophoresis, laser treatment or ultrasound treatment with elasticizing gel (in cases of tissue fibrosis).

The combination of the manual (passive) redression, where soft tissue contracture is gradually overcome by manual intervention administered by a physiotherapist, and mechanical redression with the use of a customized orthosis proceeds according to the following schema:

1) If the contracture is observed in non-joint tissues, i.e. muscles, tendons, ligaments, nerves or fascia, with no involvement of joints and joint capsules, manual intervention comprises gradual depression of the first, fourth and fifth metatarsal while providing support for the second and third metatarsal until the contractures abates.
2) If the contractures affects the capsules of tarsometatarsal joints no. I-V (the Lisfranc joint complex) the procedure follows the Kaltenborn-Evjenth convex-concave rule, i.e. it begins by relaxing the affected joint capsules and follows up with mobilization of the joints themselves. This approach protects foot joints against mechanical damage, subluxation compounded by pathological asymmetric compression of articular cartilage, excessive wear, dystrophy and degeneration. This stage is essential as otherwise the procedure might lead to compression of articular cartilage in the Lisfranc joint complex, resulting in further degenerative changes.

In either case (1 or 2), the results of manual intervention are enhanced and reinforced through the use of a specially-designed corrective orthosis. The procedure is repeated sequentially until a satisfactory therapeutic result is achieved (conditional upon the capability of the affected tissues for sustaining deformation). In selected cases, patients undergoing the abovementioned manual/mechanical treatment may also be advised to perform exercises designed to strengthen well as undergo electrostimulation muscle as follow:
the short foot muscles
the muscles of the lower leg reaching to the foot bone
the muscles do not have endings on the foot and often distant, which, through the synergy of secondary based on the irradiation of excitation will help strengthen the muscles of the foot.

In some cases the manual part of the procedure may be skipped and the orthosis applied directly. This applies to the following classes of patients:
Patients with no discernible degeneration of the musculoskeletal system, aiming to restore the physiological architecture of the foot caused by weakening of muscles stabilizing the foot e.g. through long-term use of non-physiological footwear.
Post-operative patients who have undergone surgical treatment of orthopedic deformities (e.g. HV corrective surgery), where the orthosis supports the restored transverse arch of the foot and helps ensure long-term preservation of the therapeutic outcome.

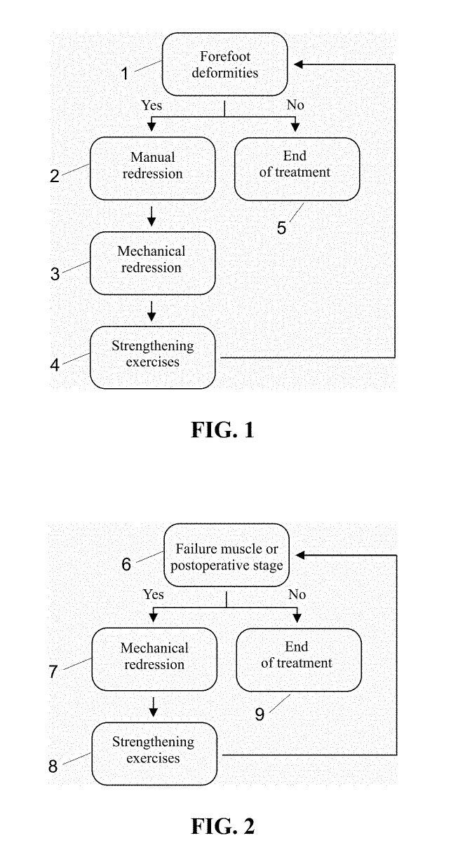

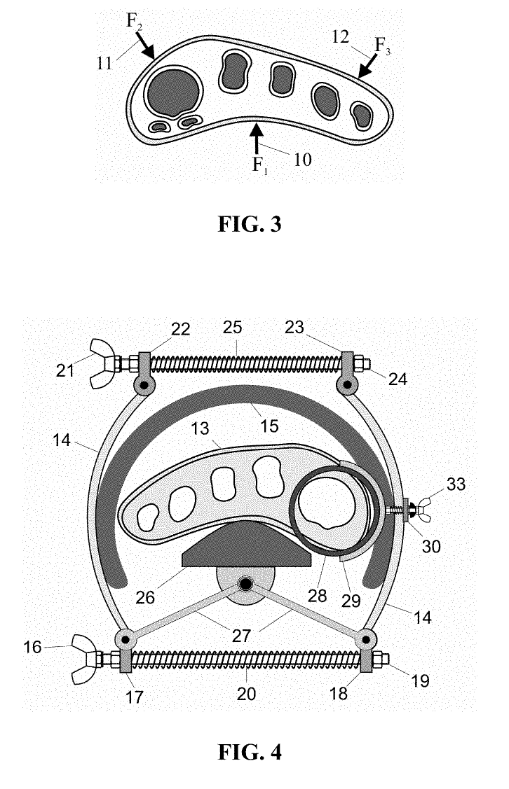

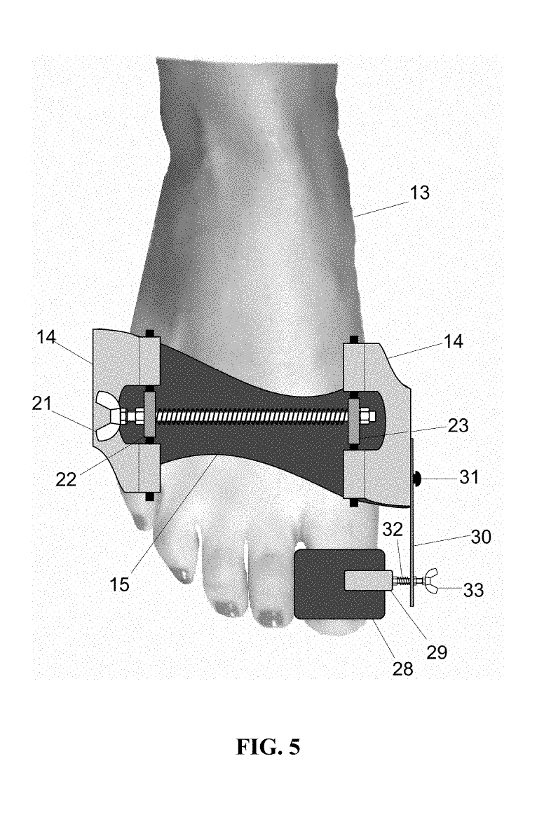

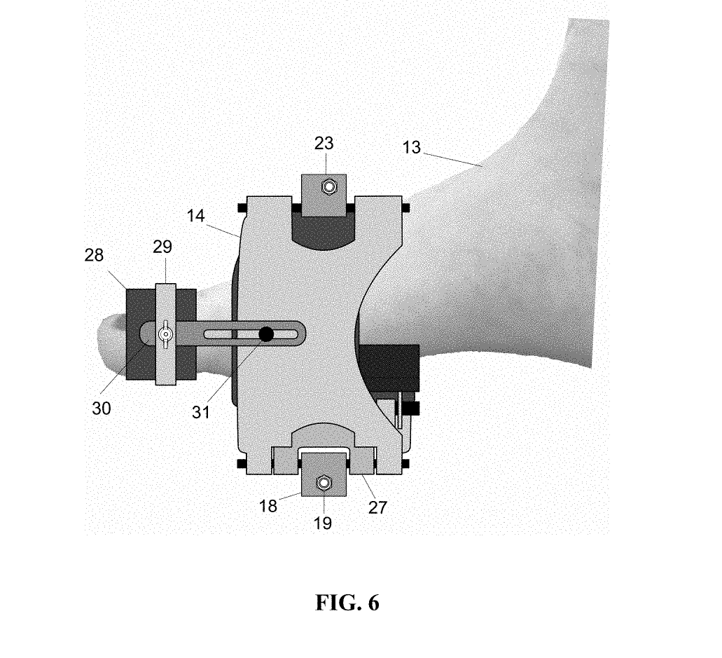

The attached figures illustrate the therapeutic procedure and technical details of the invention. FIG. 1 presents the therapeutic protocol; FIG. 2 presents the corresponding prophylactic (preventive) protocol; FIG. 3 depicts the three-force rule upon which the corrective orthosis is based, while FIG. 4 provides a technical depiction of the orthosis along with its constituent parts.

1. The manual/mechanical approach to correcting or for preventive and postoperative using is a sequential protocols in the therapeutic procedure (1-5)—for patient with transverse static flatfoot often compounded by hallux valgus to produce the desired therapeutic effect, i.e. restoration of the physiological transverse arch of the foot, elimination of hallux valgus and alleviation of pain or the prophylactic (preventive) procedure (6-9) for patients with weak stabilizing muscles and for patients after operations foot deformity, both (therapeutic and prophylactic (preventive) procedures) with using the mechanical orthosis (13-33) bases on the so-called “three forces” (F₁ vs F₂, F₃) (FIG. 3) principle (10-12).

2. With respect to the therapeutic procedure (FIG. 1) and the prophylactic (preventive) procedure (FIG. 2) according to claim 1, the manual/mechanical protocol presented in (1-5) and (6-9) is significant in that it applies to the following cases:
if contracture is observed in non-joint soft tissues, i.e. muscles, tendons, ligaments, nerves and fascia, the procedure comprises gradual depression of the mobile metatarsal bones (Ist, IVth and Vth) with a counterforce applied to the immobile metatarsal bones (IInd and IIIrd) (1-4) until the contracture is overcome and the pathology remediated (5),
if the contracture affects the capsules of tarsometatarsal joints no. I-V (the Lisfranc joint complex) the procedure follows the Kaltenborn-Evjenth convex-concave rule, i.e. it begins by stretching the affected joint capsules and follows up with mobilization of the joints themselves (1-4) to maximize the corrective effect (5). In such cases the procedure follows the scheme depicted in FIG. 1,
if no orthopedic deformation is present but the patient's stabilizing foot muscles are weakened, the mechanical orthosis may be applied prophylactically (preventively) to counteract potential pathological changes, as depicted in (6-9) in FIG. 2,
if the patient is recovering from surgery (e.g. for hallux valgus) the mechanical orthosis may be applied prophylactically (preventively) (6-9) to reinforce the result of surgical treatment. The corresponding procedure is depicted in FIG. 2.
In all of the above cases the restored transverse arch (note that no manual redress is foreseen in prophylactic (preventive) and post-operative application) is reinforced by applying a customized orthosis (13-30). Manual and mechanical redress is applied sequentially until the desired therapeutic effect has been obtained, limited by the tissues' capability for sustaining deformation. In certain cases patients may be advised to supplement the manual/mechanical protocol with additional exercises strengthening as well as electrostimulation (4) (8) of muscle as follow:
the short foot muscles
the muscles of the lower leg reaching to the foot bone
the muscles do not have endings on the foot and often distant, which, through the synergy of secondary based on the irradiation of excitation will help strengthen the muscles of the foot.

3. With regard to the design of the mechanical orthosis (the corrective apparatus) according to claim 1—the manual/mechanical approach depicted in FIG. 4, FIG. 5 and FIG. 6 is significant in that:
it depresses the first, fourth and fifth metatarsal bones while applying a counterforce to the second and third metatarsal bones. This is achieved with the use of a specially-designed orthosis, which comprises a counterforce assembly (26-27), a regulatory element (16-20) and a depressor for the first, fourth and fifth metatarsal bones (14) equipped with a screw adjustor (21-25) to allow gradual application of force in a manner consistent with the tissues' capability for sustaining deformation. All components in contact with the foot are fabricated from an elastic resin which does not irritate the skin (15).
it enables gradual restoration of the transverse arch by abducting the hallux and restoring its approximate physiological orientation through the use of an adjustable system consisting of an elastic pelote (28), a semiannular pelote mount (29), an adjustable mounting arm (30), a pelote mount with arm length adjustment capability (31), a tension control screw (32) and a screw handle (33). The pelote works by exerting an abducting force on the hallux, restoring its physiological orientation. This procedure can be applied only when hallux valgus is a result of non joint tissue contracture (phase II or early phase III, i.e. contracture of the joint capsule). In phase I restoration of the transverse arch is based on mobilizing muscles and tendons (contracture extra-articular tissues), which naturally mitigates the symptoms of hallux valgus with no need for a pelote.
